(19) United States
(12) Patent Application Publication
(10) Pub. No.: US 2004/0022668 A1
(43) Pub. Date: Feb. 5, 2004

(54) Micro-organism mail sterilizer

(76) Inventor: William J. Kitchen, Orlando, FL (US)

Correspondence Address: RICK MARTIN, PATENT LAW OFFICES OF RICK MARTIN, PC, 416 COFFMAN STREET, LONGMONT, CO, 80501, US

(21) Appl. No.: 10/299,240

(22) Filed: Nov. 19, 2002

Related U.S. Application Data
(60) Provisional application No. 60/339,614, filed on Nov. 19, 2001.
(60) Provisional application No. 60/337,212, filed on Nov. 20, 2001.

(51) Int. Cl. A61L 2/00; A62B 7/08; A61L 9/00

(52) U.S. Cl. 422/22; 422/1; 422/21; 422/28; 422/120; 422/305; 422/306

(57) ABSTRACT

A single or multiple source decontamination device for containment with a simple mailbox individual receptacle or a larger office environment receptacle. Sources such as a gaseous release, ultraviolet, infrared, microwave, ultrasonic, deionization, etc. are employed to decontaminate delivered packages within the receptacle. The device offers protection against packages potentially contaminated with bacterial or viral microorganism, anthrax being one example.

### CROSS REFERENCE APPLICATIONS

This application is a non-provisional application claiming the benefits of provisional application Nos. 60/339,614 filed Nov. 19, 2001 and 60/337,212 filed Nov. 20, 2001.

### FIELD OF THE INVENTION

The present invention relates to anti-bacterial, anti-viral mail sterilizer employing means of gas, microwave, ultraviolet, infrared, ionization, or ultrasonic emitters to kill impurities on delivered mail.

### BACKGROUND OF THE INVENTION

The present invention relates to decontamination of mail or other products. It finds particular application in conjunction with personal mail or small packages and is suitable for individual application or office environment applications.

There are various U.S. Patents that refer to various means of mailbox locking and alarms. U.S. Pat. No. 6,222,451 B1 (2001) to Leow relates to a signal device that activates an audio or visual alarm or combination of the two when a mail envelope is inserted into the mailbox. U.S. Pat. No. 6,028,517 (2000) to Sansone et al. relates to systems for indicating the deposit and withdrawal of items in a receptacle. U.S. Pat. No. 5,979,750 (1999) to Kindell relates to a computerized delivery-acceptance system for a home delivery box with an input door and an outlet door. U.S. Pat. No. 5,921,117 (1999) to Illguth describes a locking device for securing a mailbox or other receptacle. U.S. Pat. No. 5,917,411 (1999) to Baggarly describes an electronic mailbox with a keypad alarm system and a programmed timed delay system to enable those who are authorized to access the mail without triggering the alarm. U.S. Pat. No. 5,850,967 (1998) to White describes a mailbox locking mechanism. U.S. Pat. No. 5,692,674 (1997) to Wicher describes a mechanically self-locking receptacle. U.S. Pat. No. 5,586,718 (1996) to Speece describes a lock assembly for a mailbox. U.S. Pat. No. 5,476,220 (1995) to Cohoon describes a lockable mailbox apparatus that incorporates an automatically actuated latching mechanism. U.S. Pat. No. 5,407,126 (1995) to Coultas et al. describes a single-door locking mailbox.

There are many various U.S. Patents that refer to various methods of sanitization/decontamination. U.S. Pat. No. 5,374,394 (1994) to Kralovic relates to a grinder, a dilutant, and dry reagents to form an anti-microbial solution with the dilutant. U.S. Pat. No. 5,422,074 (1995) to Schmidt relates to granulating waste material and treating with heat and steam pressure. U.S. Pat. No. 4,953,732 (1990) to Cocks relates to collection and storage of contaminated body fluid waste material. U.S. Pat. No. 3,926,107 (1975) to Dunlap et al. relates to a process for rendering contents of a compactor-type solid waste and storage unit stable, nonodorous, and insect free. U.S. Pat. No. 3,636,862 (1972) to Bottas relates to a refuse compactor with an applicator to apply a treating chemical agent for the refuse. U.S. Pat. No. 3,625,433 (1971) to Moss et al. relates to a grain bin insecticide applicator. U.S. Pat. No. 3,393,825 (1968) to Clauser relates to an aerosol disinfectant bomb attached to a garbage can. U.S. Pat. No. 3,307,902 (1967) to Nardi relates to disinfecting and deodorizing cloths, linens, etc. U.S. Pat. No. 3,229,914 (1966) to Seavey relates to atomizers in the application to treatment of garbage or refuse containers. U.S. Pat. No. 2,652,173 (1953) to Farrell relates to a germicidal receptacle that projects the germicide at intervals to inhibit the growth of certain bacteria. U.S. Pat. No. 2,281,630 (1942) to Southard relates to receptacles for disposal and disinfecting of sanitary napkins. U.S. Pat. No. 1,481,685 (1924) to Burrows relates to a garbage can equipped with a removable cuspidor connected to a waste pipe and a spray nozzle for flushing. U.S. Pat. No. 1,169,302 (1916) to Triggs describes a garbage receptacle accessible from both inside and outside discharging disinfectants upon opining the receptacle door.

On Sep. 11, 2001 the terrorist attack at the New York City World Trade Center made all citizens of the United States of America highly aware of what acts of terrorism can produce. In the weeks that followed Sep. 11, 2001, the U.S. Postal Service was also attacked with a substance know as anthrax which subsequently caused illness and deaths of individuals by passing the bacteria through the mail. Such a substance as anthrax (a bacteria) can be passed directly to the receiver. Direct inhalation of anthrax spores proved to be fatal, whereas skin contact caused skin lesions. Skin exposure to anthrax spores can be a result of secondary contact either directly with the effected mail or as it may be passed from one mailing onto another (secondary contamination). Mail delivery systems were subsequently halted in many area of the United States, and large order decontamination was needed.

What is needed is a system to provide a protection scheme at the point of delivery both to individuals and small offices. The present invention will not only provide added protection by decontamination methods but also provide individuals with added assurance of a their own individual secondary protection layer.

### SUMMARY OF THE INVENTION

The main aspect of the present invention is to provide an individual(s) with a personal mail protection device to allow for decontamination of microorganisms within home mail deliveries.

Another aspect of the present invention is to provide for a larger office group protection device to allow for decontamination of microorganisms within a small office environment.

Another aspect of the present invention is to provide for a protection device with more than one decontamination component.

Another aspect of the present invention is to provide for a protection device with various combinations of decontamination devices.

Another aspect of the present invention is to provide for variable preset timing of the decontamination device(s) on-times.

Other aspects of this invention will appear from the following description and appended claims, reference being made to the accompanying drawings forming a part of this specification wherein like reference characters designate corresponding parts in the several views.

The present invention relates to the decontamination or sterilization of mail articles. However, the present invention can also relate to the application in connection with the decontamination of any product(s) that might potentially prove to have harmful microorganisms. The present invention provides an individual(s) with a personal mail microorganism decontamination system. The device allows for decontamination of bacterial or viral organisms by implementation of various decontamination components that include but are not necessarily limited to the following:

A. A gaseous spray

B. A microwave power source

C. An ultraviolet power source

D. An infrared power source

E. An ionizer source

F. An ultrasonic source

G. Combinations of the above

The present invention would provide for a decontamination source device that would allow mail (or other products) to be received within a container similar in size to a mailbox for individual mail protection or larger in size for larger groups' deliveries or package decontamination. A larger container would be similar in size to a microwave oven. Either package would be powered by either a dc or ac power source depending on installation requirements. For example, a dc source would relate easily to older outdoor installations that would be more easily installed. New outdoor installations could have either an ac or dc power option, whereas indoor installations would preferably use ac power. The device of the present invention would allow for a lock and sensor that would be pre-set so that the mail delivery person could gain access to the device (mail box or larger container). Upon the delivery person closing the door, the lock would be activated until unlocked by the owner. Such a locking mechanism is known in the prior art. The present invention also allows for a door opening/closing sensor in lieu of a locking mechanism. With the closure and locking (or simply sensing closure) of the device door, a timer would be activated. The timer would count down a set time period. At the end of the set time period logic circuitry would trigger one or more events depending on the number of installed decontamination means. For example, if only a decontamination gas were installed, the timeout would activate a release of the gas for a set time. If a gas and an alternate decontamination source (ultraviolet, infrared, microwave, deionizer, ultrasonic, etc.) were installed, then the timeout would activate a series of events to have a timed release of the gas, activate the alternate decontamination source for a specified time period and then reset. If more than one alternate decontamination source were installed, then the steps would be added to the series of events to activate all installed decontamination sources in series.

### BRIEF DESCRIPTION OF THE DRAWINGS

FIG. 1A is a perspective view of a mailbox sterilizer with an installed gas release mechanism.

FIG. 1B is a perspective view of a mailbox sterilizer with both a gas release mechanism and an alternate decontamination source.

FIG. 1C is an alternate embodiment of the present invention showing a perspective view of a mailbox sterilizer with a gas release mechanism in the mounting pole and a alternate decontamination source in the mailbox.

FIG. 1D is an alternate embodiment of the present invention showing a perspective view of the mailbox sterilizer with an owner bypass activation button.

FIG. 2A is a perspective view of a larger decontamination device with a gas release mechanism and an alternate decontamination source.

FIG. 2B is a perspective view of a larger decontamination device with a gas release mechanism and two alternate decontamination sources.

FIG. 3 is a flow chart of the process of the present invention relating to the steps of decontamination.

Before explaining the disclosed embodiment of the present invention in detail, it is to be understood that the invention is not limited in its application to the details of the particular arrangement shown, since the invention is capable of other embodiments. Also, the terminology used herein is for the purpose of description and not of limitation.

### DETAILED DESCRIPTION OF DRAWINGS

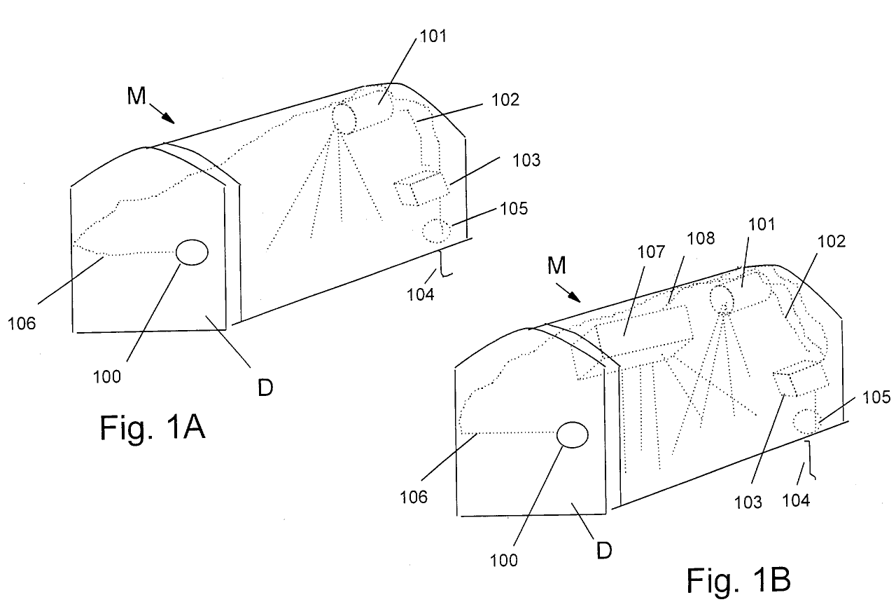

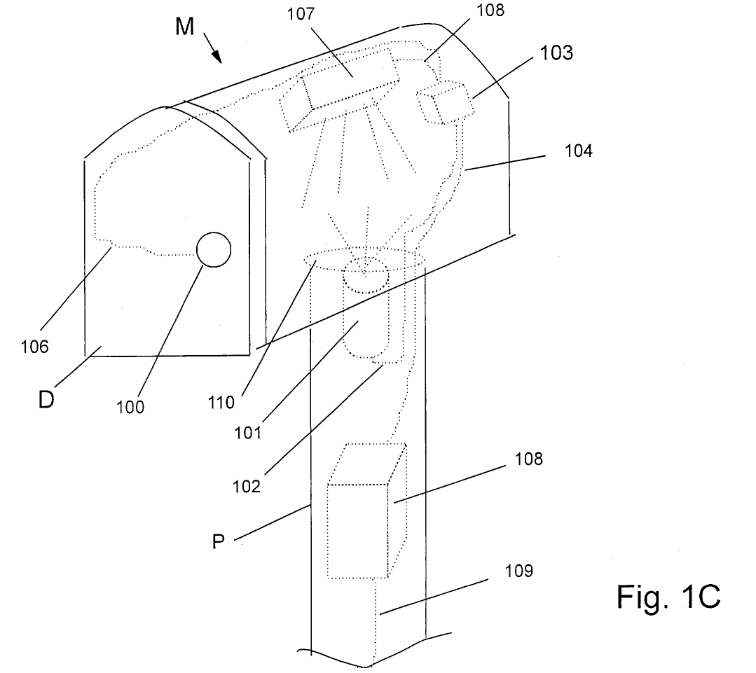

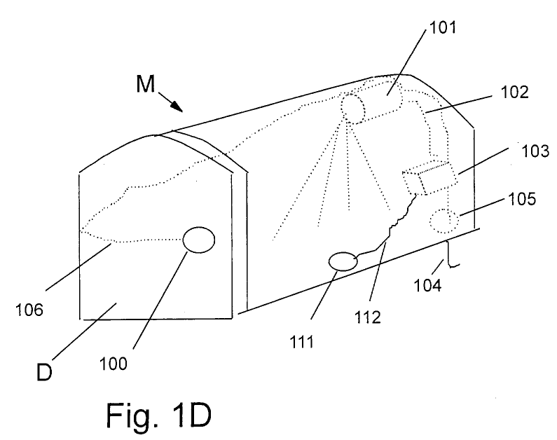

FIG. 1A is a perspective view of a mailbox sterilizer M with an installed gas release mechanism 101. A door lock and opening/closing sensor mechanism 100 senses when the door D is shut after a delivery. The lock mechanism can be eliminated so that only the opening/closing sensor is installed. A logic block/timer 103 is attached to the door lock and opening/closing sensor mechanism 100 by lock/sensor mechanism wire circuitry 106, to the gas release mechanism 101 by gas release mechanism wire circuitry 102 and to an external power source by power wire circuitry 104, which is available through an exterior mailbox hole 105. Upon the logic/timer 103 sensing that the door lock and opening/closing sensor mechanism 100 has been shut (activated), which will occur after a delivery, the logic/timer 103 will determine that a gas release mechanism 101 is installed. The logic/timer 103 will start a delay timeout, after which the logic/timer 103 will activate a gas release for a specified period of time from the gas release mechanism 101 in order to decontaminate the delivered material. After the gas release time the timing cycle will stop. When the door lock and opening/closing sensor mechanism 100 senses that the door D is again opened, the logic/timer 103 will reset until the door lock and opening/closing sensor mechanism 100 is re-activated by locking/closing subsequent to another delivery. It should be noted that although FIG. 1A shows a gas release mechanism 101, an alternate decontamination source could also be substituted. It should also be noted that although the power wire circuitry 104 leads to an exterior port for input power, a dc source (battery) is an option that might be installed internal to the mailbox (not shown). Depending on postal delivery requirements, a lock and opening/closing mechanism may not have the lock included. A further option could be a gas powered control and activation system. A further option could be an indicator light on the mailbox to indicate a decontamination cycle is in progress, thereby alerting the owner not to open the mailbox. Some decontamination devices may have an ON period, a saturation period and/or a purge period, thereby providing a decontamination cycle. The decontamination cycle for certain devices such as a microwave transmitter would simply be an ON and OFF state.

FIG. 1B is a perspective view of a mailbox sterilizer M similar to FIG. 1A but with both a gas release mechanism 101 and an alternate decontamination source 107 which would together act as dual decontamination means. An alternate decontamination source 107 such as ultraviolet, infrared, microwave, deionizer, ultrasonic, is connected to the logic/timer 103 by means of alternate decontamination source circuitry 108. Upon the logic/timer 103 sensing that the door lock mechanism 100 has been shut (activated), which will occur after a delivery, the logic/timer 103 will determine the that a gas release mechanism 101 and a alternate decontamination source 107 are both installed, start a delay timeout, after which the logic/timer 103 will activate a gas release for a specified period of time from the gas release mechanism 101. Next the logic/timer 103 will activate the alternate decontamination source 107 (microwave, ultraviolet, infrared, ultrasonic, deionizer, etc.) in order to decontaminate the delivered material. After the two-step decontamination is over, the timing cycle will stop. When the door lock mechanism 100 senses that the door D is again opened the logic/timer 103 will reset until the door lock mechanism 100 is re-activated by locking subsequent to another delivery. It should be noted that although FIG. 1A shows only two decontamination sources, a design with more than two decontamination sources could be substituted. It should also be noted that although the power wire circuitry 104 leads to an exterior port for input power, a dc source (battery) is an option that might be installed internal to the mailbox (not shown).

FIG. 1C is an alternate embodiment of the present invention similar to FIGS. 1A, 1B but showing a perspective view of a mailbox sterilizer M with a gas release mechanism 101 in a mounting pole P and an alternate decontamination source 107 in the mailbox. The power wire circuitry 104 and the gas release mechanism wire circuitry 102 utilize a top pole hole 110 for routing. An ac-to-dc converter 108 would convert ac power, entered into the pole P by ac power cable 109, into dc power for the logic/timer 103 etc. The ac-to-dc power converter 108 might also be replaced (not shown) with a dc-power source (battery) installed within the pole P for installations not easily adapted to ac power. An indicator could be supplied to indicate any lapse in the power source, whether terrorist caused or otherwise. A special key or code on a pad would be needed to reset this indicator.

FIG. 1D is an alternate embodiment of the present invention showing a perspective view of the mailbox sterilizer M with an owner bypass activation button 111 which is attached to the logic/timer 103 with bypass activation wiring 112. The owner bypass activation button 111 would function to allow the owner to open and close the front door D without activation of any decontamination device. The preferred embodiment would be a key pad with a secret code known only by the owner. A simple key could also be used.

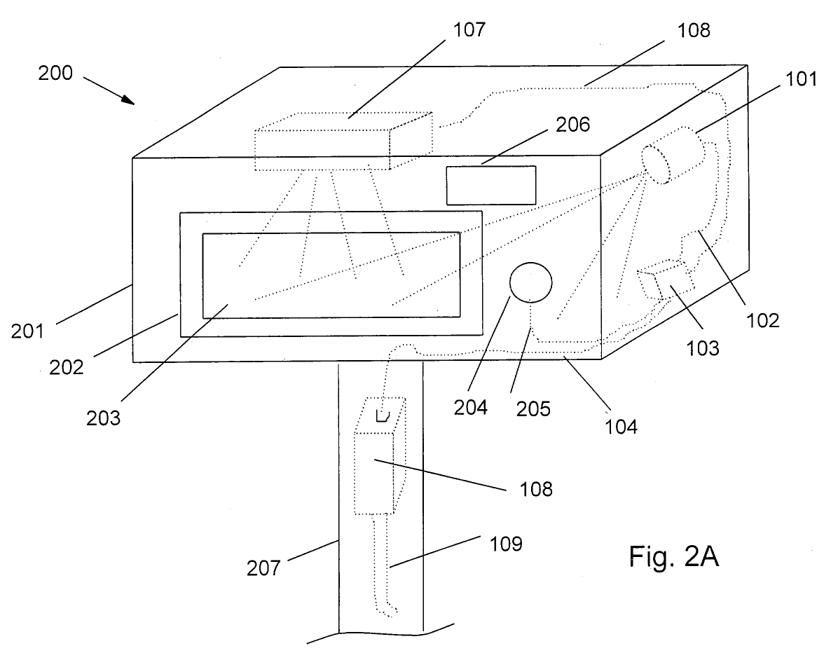

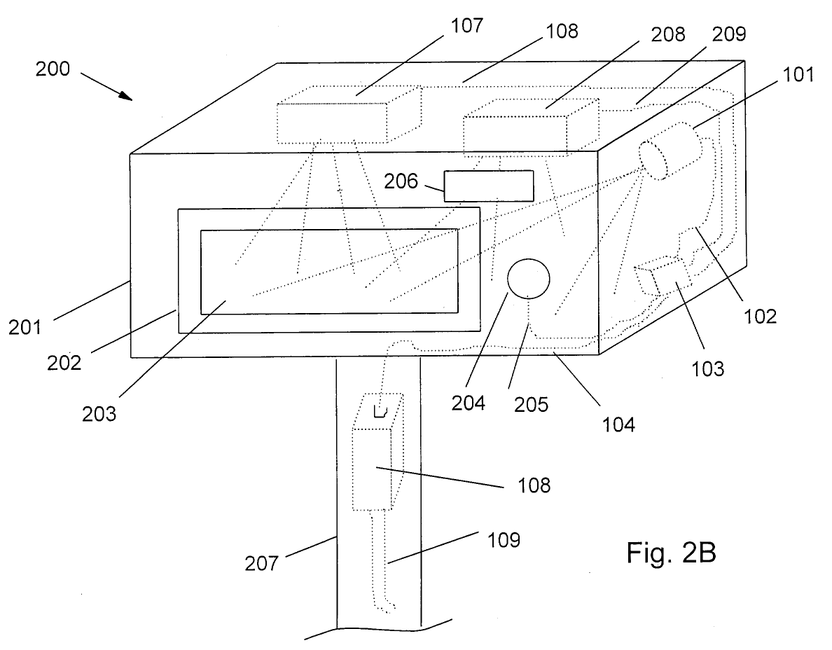

FIG. 2A is a perspective view of a larger decontamination device 200 with a gas release mechanism 101 and an alternate decontamination source 107. A larger receptacle 201 contains an opening door 202 that has a glass/metal window 203 similar to a microwave oven for viewing internal contents. A door lock and opening/closing sensor mechanism 204 is logically connected to the logic/timer 103 by door lock and opening/closing sensor mechanism circuitry 205. A control panel 206 would be available for controlling the logic/timer 104 cycles beyond a minimal set point. Logically FIG. 2A is similar to FIG. 1B with both a gas release mechanism 101 and an alternate decontamination source 107 which would together act as dual decontamination means. An alternate decontamination source 107 such as ultraviolet, infrared, microwave, deionizer, ultrasonic, is connected to the logic/timer 103 by means of alternate decontamination source circuitry 108. Upon the logic/timer 103 sensing that the door mechanism 204 has been shut (activated), which will occur after a delivery, the logic/timer 103 will determine that a gas release mechanism 101 and an alternate decontamination source 107 are both installed, start a delay timeout, after which the logic/timer 103 will activate a gas release for a specified period of time from the gas release mechanism 101. Next, the logic/timer 103 will activate the alternate decontamination source 107 (microwave, ultraviolet, infrared, ultrasonic, deionizer, etc.) in order to decontaminate the delivered material. After the two-step decontamination is over, the timing cycle will stop. When the door lock and opening/closing sensor mechanism 204 senses that the door is again opened, the logic/timer 103 will reset until the door lock and opening/closing sensor mechanism 204 is reactivated by locking subsequent to another delivery. The locking part of the door lock and opening/closing mechanism 204 may be omitted from installation in lieu of postal delivery requirements. It should be noted that although FIG. 2A shows the power wire circuitry 109 leads to an exterior port for input power through a pole mount 207, it could easily have the ac-to-dc converter 108 internally installed within receptacle 201 such that a standard ac power plug (not shown) leaves the receptacle 201. The owner can bypass activation of decontamination devices prior to opening the door 202 by using the control panel 206.

FIG. 2B is a perspective view of a larger decontamination device 200 and is similar to FIG. 2A but with a gas release mechanism 101 and two alternate decontamination sources 107, 208. Thus, the installation of the first decontamination source 107 and the second decontamination source 208 would have many combinations of sources previously described (microwave, ultraviolet, infrared, ultrasonic, deionization etc.). The second alternate decontamination source 208 is connected to the logic/timer 103 with second alternate decontamination circuitry 209. Upon the logic/timer 103 sensing that the door mechanism 204 has been shut (activated), which will occur after a delivery, the logic/timer 103 will determine that a gas release mechanism 101 and two alternate decontamination sources 107, 208 are all installed, start a delay timeout, after which the logic/timer 103 will activate a gas release for a specified period of time from the gas release mechanism 101. Next, the logic/timer 103 will activate the alternate decontamination source 107 (microwave, ultraviolet, infrared, ultrasonic, deionizer, etc.) followed by activation of the second decontamination source 208 in order to decontaminate the delivered material. After the three-step decontamination is over, the timing cycle will stop. When the door lock and opening/closing sensor mechanism 204 senses that the door is again opened, the logic/timer 103 will reset until the door lock and opening/closing sensor mechanism 204 is re-activated by locking subsequent to another delivery. The locking part of the door lock and opening/closing mechanism 204 may be omitted from installation in lieu of postal delivery requirements. It should be noted that a larger decontamination device 200 can be designed to contain more than the three sources shown (gas and two alternative sources). An equivalent to any doors of the above embodiments would include sliding members, rolling members, clamshell members and any mechanical members functioning to prevent an item from entering an enclosure.

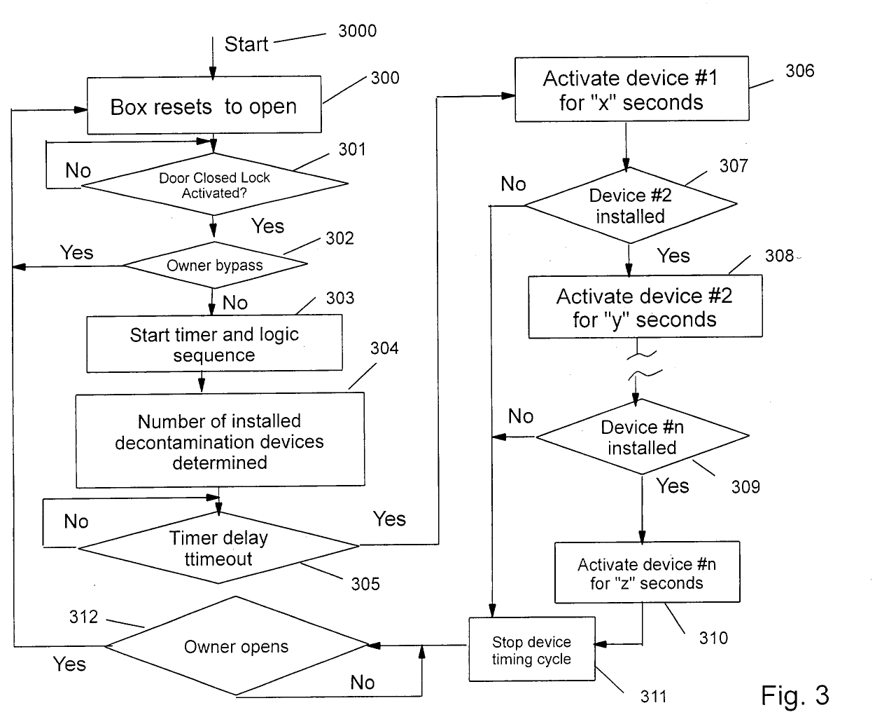

FIG. 3 is a flow chart of the process of the present invention relating to the steps of decontamination as described in the receptacles of FIGS. 1A through 2B. At the start 3000 of the cycle a box (receptacle) is in an “open” reset condition 300. The door closure and lock activation is sensed 301. If the door closure and lock is not activated it continues sensing. If a bypass button were installed, and if the owner had bypassed activation of decontamination devices 302, the box resets to open 300 and the timing sequence is not started. If the owner did not bypass activation 302, the timer and logic sequence is started 303. The number of installed decontamination devices “n” is determined 304 and a timer delay timeout 305 is begun. This is to insure a delay prior to activating any decontamination device. Next, device #1 is activated for “x” seconds 306. Next, it is determined if another decontamination device is installed 307. If no other device is installed, the device timing cycle is stopped 311. If a second decontamination device is installed the device #2 is activated for “y” seconds 308. If no other decontamination device is installed the device timing cycle is stopped 311. The procedure continues until the last “n” installed device is determined 309. Once the last device is reached, it is activated 310 followed by the device timing cycle stopped 311. When the device timing cycle is finally stopped 311, the logic cycles until the owner opens the receptacle 312. Once the receptacle is open, the box resets to open 301 awaiting another delivery, thus completing one full decontamination loop.

Although the present invention has been described with reference to preferred embodiments, numerous modifications and variations can be made and still the result will come within the scope of the invention. No limitation with respect to the specific embodiments disclosed herein is intended or should be inferred.

I claim:

1. An anti-terrorist mailbox comprising:
a housing having a door to receive an item;
said door having an open and a closed position;
a first decontamination device associated with the mailbox to decontaminate the item;
a sensor to detect the position of the door;
a controller to receive a signal from the sensor that the door has been opened; and
said controller having a control circuit to initiate a decontamination cycle of the first decontamination device upon receiving the signal from the sensor.

2. The apparatus of claim 1 further comprising an indicator to indicate not to open the door during the decontamination cycle.

3. The apparatus of claim 1 further comprising a door lock controlled by the controller to lock the door when the first decontamination device is in the decontamination cycle.

4. The apparatus of claim 3, wherein the control circuit further comprises a timer to control the decontamination cycle for the first decontamination device.

5. The apparatus of claim 1 wherein the decontamination cycle further comprises an ON time and a saturation time.

6. The apparatus of claim 1 further comprising a second decontamination device, wherein the controller controls a decontamination cycle for each of the first and the second decontamination devices.

7. The apparatus of claim 6 further comprising a door lock to lock the door when either the first or the second decontamination device is in a decontamination cycle.

8. The apparatus of claim 7 further comprising an owner bypass switch which allows the door to be opened without an initiation of a decontamination cycle.

9. The apparatus of claim 8 further comprising an indicator to indicate not to open the door during a decontamination cycle.

10. The apparatus of claim 6, wherein the first decontamination device further comprises a gas release system, and the second decontamination device further comprises a microwave device.

11. The apparatus of claim 6, wherein the controller is electronic, and a power source is supplied to the controller.

12. The apparatus of claim 11, wherein the power source is an ac/dc converter located in a pole associated with the mailbox.

13. The apparatus of claim 11, wherein the power source is a battery located in a pole associated with the mailbox.

14. The apparatus of claim 11 further comprising an indicator to show any loss of power.

15. The apparatus of claim 1, wherein the first decontamination device is located in the housing.

16. An anti-terrorist mailbox comprising:
a housing having a closure means functioning to enable/disable a receipt/withdrawal of mail;
an n number of decontamination devices each functioning to decontaminate the mail in the housing;
a closure sensor means functioning to send a signal indicating an OPEN/SHUT condition of the closure means; and
a controller means functioning to receive the signal and operate the n number of decontamination devices in a predetermined manner.

17. The apparatus of claim 16, wherein the controller means further comprises a circuit to control a lock for the closure means.

18. The apparatus of claim 16 further comprising a power source means associated outside of the housing.

19. The apparatus of claim 16, wherein at least one of the decontamination devices is located in the housing.

20. A method to decontaminate mail comprising the steps of:
sensing an opening of a door on a mailbox;
activating a decontamination device in a predetermined manner when the opening of the door is sensed; and
indicating when the decontamination device is ON.
